(19) United States
(12) Patent Application Publication
(10) Pub. No.: US 2009/0281582 A1
(43) Pub. Date: Nov. 12, 2009

(54) INSTRUMENT FOR THE REDUCTION OF A ROD INTO POSITION IN A PEDICLE SCREW

(76) Inventors: Raul Villa, Pflugerville, TX (US); Charles R. Forton, Leander, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 12/117,484

(22) Filed: May 8, 2008

(51) Int. Cl. A61B 5/00; A61B 17/00

(52) U.S. Cl. 606/86 A; 606/207; 606/206

(57) ABSTRACT

Instruments for reducing spinal stabilization rods into position in pedicle screws or other bone anchors placed on a spine. In some embodiments, instruments are provided which include inner and outer extensions adapted for engaging, respectively, pedicle screws and rods. Handles of the instrument can engage a parallel action compressor which actuate the extensions. The handles may be offset from the extensions by a distance in a direction along a longitudinal axis of the extensions, in a direction perpendicular thereto, or a combination thereof. Instruments of some embodiments can include locking, or ratchet, mechanisms extending between the handles. A biasing member may bias the handles apart from each other and assist in engaging the locking or ratchet mechanism. Surgical instrument kits including rod reduction instruments are provided by some embodiments. According to some embodiments, methods for reducing spinal stabilization rods into their desired positions are also provided.

### TECHNICAL FIELD

Embodiments of the disclosure relate generally to instruments for spinal surgery and more particularly to instruments for reducing rods of spinal stabilization systems into position in pedicle screws.

### BACKGROUND

Modern spine surgery often involves the use of spinal stabilization procedures to correct or treat various acute or chronic spine disorders or to support the spine. Spinal stabilization systems may help, for example, to stabilize the spine, correct deformities of the spine such as spondylolisthesis or pseudarthrosis, facilitate fusion, or treat spinal fractures. Some spinal stabilization systems may provide rigid support for the affected regions of the spine such as when they are used in conjunction with a vertebral body fusion procedure. Some spinal stabilization systems can limit movement in the affected regions in virtually all directions, again, such as when used in conjunction with a vertebral fusion procedure. Dynamic spinal stabilization systems can be provided which can allow the patient a greater range of motion (in terms of flexion, extension, or both) and can better match the patient's anatomy than some spinal stabilization systems used to provide static support. Dynamic stabilization systems can be used in scenarios in which vertebral body fusion is not desired, in which vertebral body (re)alignment is desired, in which it is desired to support or strengthen degraded, diseased, damaged, or otherwise weakened portions of the spine.

Often, spinal stabilization systems include rods which can bear a portion of the forces that would otherwise be transmitted along the spine. These rods may be implanted in pairs or in other numbers along portions of the spine of interest. Some stabilization systems support a portion of the spine including only two vertebrae (and associated anatomical structures) while some stabilization systems support portions of the spine extending beyond two vertebrae. Stabilizations systems can be used to support portions of the lumbar spine although stabilization systems can be used to support other portions of the spine such as the thoracic spine. Regardless of the number of rods implanted, or the portion of the spine in which they may be implanted, the rods can be attached to one or more vertebrae of the spine to provide support to, stabilize, align, or otherwise treat the region of the spine of interest. Many times, surgical personnel use one or more anchor systems to attach the rods to one or more vertebrae. One such anchor system includes pedicle screws constructs which define slots, keyways, grooves, apertures, or other features for accepting and retaining stabilization rods (static, dynamic, or both). In many pedicle screw constructs, pedicle screws are placed in vertebrae selected by surgical personnel.

Sometimes it happens that a rod (or more than one rod) remains proud of its desired or final position in the rod slot of the screw head by some height or distance. Such scenarios include surgical procedures in which it is desired to anchor a rod to more than one vertebra. One such scenario can occur when pedicle screws have been implanted in two vertebrae and it is desired to anchor a rod to a third vertebra lying between the two vertebrae. In this, and other scenarios, a rod reduction instrument can be navigated to the implant site by surgical personnel to correct this situation by urging the rod into position in the pedicle screw. When surgical personnel are using previously available rod reduction instruments, their view of the surgical site can be blocked by the body of the instrument. In other situations, the actuation handles of the instrument rotate into the line of sight of the surgical personnel. As they attempt to reduce the rod into its desired position and lock the rod in place, surgical personnel sometimes cannot see portions of the surgical site or spinal stabilization system. In some scenarios, reduced visibility of the implant site can result in slower, less efficient, and less accurate surgical results than desired. Yet, with previously available rod reduction instruments, little can be done to aid surgical personnel in this situation. The situation can be aggravated when the patient is abnormally large.

### SUMMARY

Embodiments of the present disclosure provide rod reduction instruments for spinal stabilization systems that eliminate, or at least substantially reduce, the shortcomings of prior art rod reduction instruments.

Various embodiments provide rod reduction instruments for use with pedicle screws and other bone anchors to improve visibility of the implant site during implant procedures. In some embodiments, the handles of the extension can have a parallel action, with extensions perpendicular to the parallel action of the handles. When squeezed, the handles can push one extension through the other. One of the extensions can have geometry corresponding to that of the pedicle screws and symmetric collapsible slots associated with the mating geometry to aid in grasping the pedicle screw.

The other extension can form a cylinder surrounding the first extension and can include features for collapsing the end of the first extension as the handles are compressed. The end of the second extension can have geometry corresponding to a rod to be seated in the pedicle screw. Both extensions can define cannulas. The cannula of the first extension can accept a setscrew and locking driver whereas the cannula of the second extension can accept the first extension.

Various embodiments provide instruments, instrument kits, and methods for reducing rods of spinal stabilization systems into place. Embodiments include an instrument for the reduction of a rod into position in a pedicle screw. The instrument can comprise an inner extension, an outer extension, a first handle and a second handle. The inner extension can have proximal and distal ends with the distal end of the inner extension adapted to engage the pedicle screw. The outer extension can be positioned about the inner extension and have proximal and distal ends and a longitudinal axis. The distal end of the outer extension can be adapted to engage the rod. The first handle can have proximal and distal ends, with the distal end of the first handle being coupled with the proximal end of the inner extension. The second handle can have proximal and distal ends with the distal end of the second handle being coupled with the proximal end of the outer extension. The handles can extend generally perpendicular from the respective extensions and be operatively coupled to form an actuator for urging the extensions in a direction relative to each other along the longitudinal axis. The handles can be offset from the proximal end of at least one of the extensions.

The offset can be greater than about half an inch in a direction parallel to the longitudinal axis. The offset can be about two and one half inches in a direction perpendicular to the longitudinal axis. The instrument can have a locking mechanism extending between the handles. The locking mechanism can include a ratchet with a toothed member extending between the first and second handles. The locking mechanism can include a pawl operatively coupled to one of the handles to engage the toothed member to prevent the handles from moving apart from one another. The instrument can include a biasing member coupled to the handles to urge the handles apart. The biasing member can be a leaf spring positioned to urge the pawl to engage the toothed member. The leaf spring can extend beyond the end of one of the handles to allow a user to disengage the pawl from the toothed member. The inner extension can define a passage to allow a compression member to be inserted through the passage. The instrument can include a parallel action compressor.

Embodiments include a kit for the reduction of a rod into position in a pedicle screw. The kit can include a rod reduction instrument, a set of pedicle screws, and a set of rods. The kit can include a set of setscrews for locking a rod in position in a pedicle screw. The inner extension of the rod reduction instrument can have a cannulation for accepting the setscrew. The kit can include a locking driver for locking the rod in position in the pedicle screw using a setscrew. The instrument can include an offset in a direction parallel to a longitudinal axis of the instrument, perpendicular to the longitudinal axis, or a combination thereof.

Embodiments include an instrument for the reduction of a rod into position in a pedicle screw. The instrument can comprise a female extension, a male extension, a first handle and a second handle. The female extension can have proximal and distal ends with the distal end of the female extension adapted to engage the pedicle screw. The male extension can be positioned about the female extension and have proximal and distal ends and a longitudinal axis. The distal end of the male extension can be adapted to engage the rod. The first handle can have proximal and distal ends, with the distal end of the first handle being coupled with the proximal end of the female extension. The second handle can have proximal and distal ends with the distal end of the second handle being coupled with the proximal end of the male extension. The handles can extend generally perpendicular from the respective extensions and be operatively coupled to form an actuator for urging the extensions in a direction relative to each other along the longitudinal axis. The handles can be offset from the proximal end of at least one of the extensions.

These, and other, aspects will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the disclosure, and the disclosure includes all such substitutions, modifications, additions, or rearrangements.

### BRIEF DESCRIPTION OF THE DRAWINGS

A more complete understanding of the disclosure and the advantages thereof may be acquired by referring to the following description, taken in conjunction with the accompanying drawings in which like reference numbers generally indicate like features and wherein:

FIG. 1 illustrates a spinal stabilization system including rods and pedicle screws according to some embodiments.

FIG. 2 illustrates a rod reduction instrument being used in a rod reduction procedure according to some embodiments.

FIG. 3 illustrates a rod reduction instrument according to some embodiments.

FIG. 4 illustrates a handle of a rod reduction instrument of some embodiments.

FIG. 5 illustrates a portion of a handle of a rod reduction instrument of some embodiments.

FIG. 6 illustrates a second handle of a rod reduction instrument of some embodiments.

FIG. 7 illustrates a portion of a second handle of a rod reduction instrument of some embodiments.

FIG. 8 illustrates a cross sectional view of a locking mechanism for a pair of handles of a rod reduction instrument of some embodiments.

FIG. 9 illustrates a female bar of a rod reduction instrument of some embodiments.

FIG. 10 illustrates a male bar of a rod reduction instrument of some embodiments.

FIG. 11 illustrates a biasing member of a rod reduction instrument of some embodiments.

FIG. 12 illustrates a portion of a biasing member of a rod reduction instrument of some embodiments.

FIG. 13 illustrates a second biasing member of a rod reduction instrument of some embodiments.

FIG. 14 illustrates a portion of a second biasing member of a rod reduction instrument of some embodiments.

FIG. 15 illustrates another portion of a biasing member of a rod reduction instrument of some embodiments.

FIG. 16 illustrates an offset arm of a rod reduction instrument of some embodiments.

FIG. 17 illustrates another offset arm of a rod reduction instrument of some embodiments.

FIG. 18 illustrates an inner extension distal end of a rod reduction instrument of some embodiments

### DETAILED DESCRIPTION

Various embodiments of the disclosure are illustrated in the FIGURES, like numerals being used to refer to like and corresponding parts of the various drawings. Embodiments of the disclosure provide spinal stabilization system rod reduction instruments and methods.

As used herein, the terms “comprises,” “comprising,” “includes,” “including,” “has,” “having” or any other variation thereof, are intended to cover a non-exclusive inclusion. For example, a process, process, article, or apparatus that comprises a list of elements is not necessarily limited only those elements but may include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, “in one embodiment”.

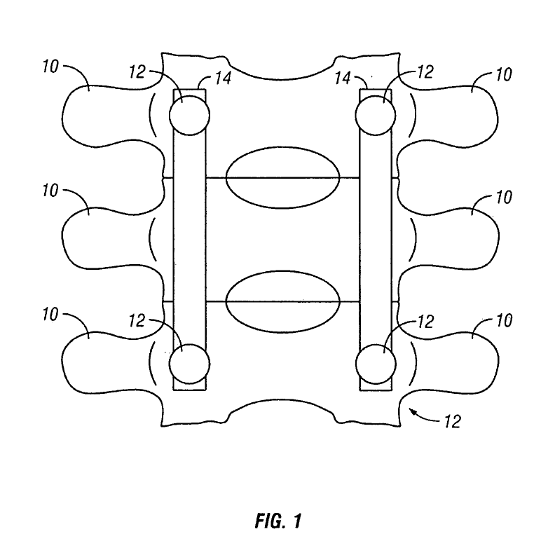

FIG. 1 illustrates a spinal stabilization system secured to several vertebrae 10 with an anchor system. The spinal stabilization system shown in FIG. 1 can be implanted with a posterior approach in which an incision is made in the patient. An anchor system can then be implanted in the patient's spine and spinal stabilization systems can be placed in one or more of the anchor systems. The rods may then be secured to the anchor systems and, if necessary, reduced into their final positions in the anchor systems. The rods may then be locked to the anchor systems and any instruments used to implant the spinal stabilization system can be removed from the patient. The surgical site may then be closed.

In various embodiments, pedicle screws 12 can secure rods 14 in place to support the spine. Only one pair of stabilization rods 14 is shown in FIG. 1. However, one skilled in the art will appreciate that different numbers of rods 14 may be utilized in various spinal procedures. As illustrated in FIG. 1, rods 14 can be fixed to selected vertebrae 10 of the spine laterally on opposite sides of the spine utilizing pedicle screws 12.

In some embodiments, any type of anchor system could be used instead of, or in addition to, pedicle screws 12. Anchor systems which can be used include pedicle screws 12, hooks, wires, etc. Pedicle screws 12 and rods 14 can be made from biocompatible material(s). Examples of biocompatible materials include titanium, stainless steel, and any suitable metallic, ceramic, polymeric, and composite materials.

The spinal stabilization system illustrated in FIG. 1 can be installed posterior to the spine, typically with the rods 14 extending parallel to the longitudinal axis of the spine lying in the mid-sagittal plane. According to some embodiments, the system can include additional rods 14 positioned further superior or inferior along the spine, with additional rods 14 being dynamic stabilization rods such as rods 14, or other types of non-dynamic, or rigid, rods 14. It should be understood that various spinal stabilization system may also include suitable transverse rods or cross-link devices that help protect the supported portion of the spine against torsional forces or movement. Some possible examples of suitable cross-link devices are shown in co-pending U.S. patent application Ser. No. 11/234,706, entitled “Apparatus And Methods For Spinal stabilization system With Variable Link Mechanism”, filed on Nov. 23, 2005 by Robert J. Jones et al., the entire contents of which are incorporated herein as if set forth in full. Other cross-link devices or transverse rods may also be employed. According to some embodiments, rods 14 can be configured to possess sufficient column strength and rigidity to protect the supported portion of the spine against lateral and torsional forces or movement. In some cases the vertebrae Rod reduction instruments of various embodiments can be used in scenarios involving the spinal stabilization system illustrated by FIG. 1 as well as other spinal stabilization systems to move a pedicle screw and hence vertebra relative to a rod to seat the rod in the pedicle screw).

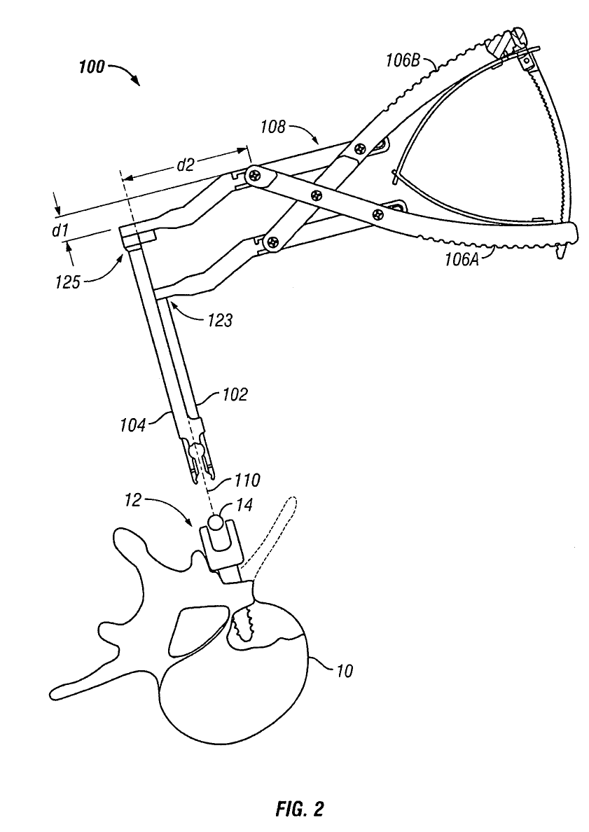

With reference now to FIG. 2, FIG. 2 illustrates rod reduction instrument 100 in use during a rod reduction procedure. FIG. 2 illustrates vertebrae 10 with pedicle screw 12 implanted therein and rod 14 proud of its intended position in pedicle screw 12 by height h1 (the distance of rod 14 from a position at rest in pedicle screw 12, not labeled). By the term “proud” it can be meant that rod 14 is some distance away from the bottom (as viewed in FIG. 2) of pedicle screw 12: Rod reduction instrument 100 can include inner extension 102, outer extension 104, handles 106, parallel action compressor 108, etc. Inner extension 102 and outer extension 104 can be annular (or have other cross sectional shapes), coaxial, and can have distal ends adapted to engage, respectively, pedicle screw 12 and rod 14 such that inner and outer extensions 102 and 104 can urge pedicle screw 12 and rod 14 together during rod reduction procedures. Outer extension 104, of some embodiments, can include features to radially compress the distal end of inner extension 102, thereby assisting inner extension 102 in grasping pedicle screw 12 and retaining pedicle screw 12 while rod 14 is reduced into position. Inner and outer extensions 102 and 104 can be operatively coupled to an actuator such as parallel action compressor 108. Parallel action compressor 108 can be operatively coupled to handles 106. However, those skilled in the art will appreciate that many types of actuation systems can be employed in lieu of parallel action compressor 108.

Longitudinal axis 110, along which inner and outer extensions 102 and 104 can be oriented, is also shown in FIG. 2. During rod reduction procedures, surgical personnel can align inner extension 102 with rod 14 so that features such as a slot, recess, keyway, groove, etc. on inner extension 102 will receive rod 14 when surgical personnel advance instrument 100 along longitudinal axis 110. Surgical personnel can then advance inner and outer extensions 102 and 104 along longitudinal axis 110 toward pedicle screw 12 and rod 14 until the distal end of inner extension 102 contacts pedicle screw 12.

Surgical personnel can urge handles 106 together to cause handles 106 and parallel action compressor 108 to operate to urge inner and outer extensions 102 and 104 in directions opposite each other along longitudinal axis 10. Surgical personnel can urge handles 106 together while maintaining contact between pedicle screw 12 and inner extension 102. As surgical personnel urge handles 106 together, the distal end of outer extension 104 can begin radially compressing the distal end of inner extension 102 thereby causing inner extension 102 to grasp pedicle screw 12. As outer extension 104 continues translating along longitudinal axis 110, the distal end of outer extension 104 can contact rod 14 and urge it into its desired position in pedicle screw 12. A setscrew or locking or compression member may then be slid through a longitudinal cannula defined by the body of inner extension 102. The compression member can then be used to lock rod 14 into position in pedicle screw 12. Surgical personnel may then urge handles 106 apart (or allow a biasing member to do so) causing outer extension 104 to translate back along longitudinal axis 110 relative to inner extension 102, thereby releasing the grasp which inner extension 102 had on pedicle screw 12. Surgical personnel may then translate instrument 100 away from pedicle screw 12.

FIG. 2 also illustrates offset distances d1 and d2 which can extend the overall distance between handles 106 and inner and outer extensions 102 and 104 thereby allowing surgical personnel improved visibility of the distal end of instrument 100, pedicle screw 12, rod 14, and the overall surgical site during all phases of rod reduction procedures. Offset distance d1 can be along a direction parallel to longitudinal axis 110 while offset distance d2 can be along a direction perpendicular to longitudinal axis 110. Those skilled in the art will understand that offsets d1 and d2 can be provided by a variety of differently shaped components of instrument 100 and, when used in conjunction with each other, can provide an offset in a direction diagonal to longitudinal axis 110. Offsets d1 and d2 each individually, and taken together, can allow surgical personnel who may be grasping handles 106 to peer around, over, or beyond the proximal ends 123 and 125 of inner and outer extensions 102 and 104 to view pedicle screw 12 and rod 14 even when inner and outer extensions 102 and 104 engage the same as when surgical personnel are reducing rod 14 into position. With improved visibility of the surgical site, according to some embodiments, surgical personnel can operate more quickly, efficiently, and accurately.

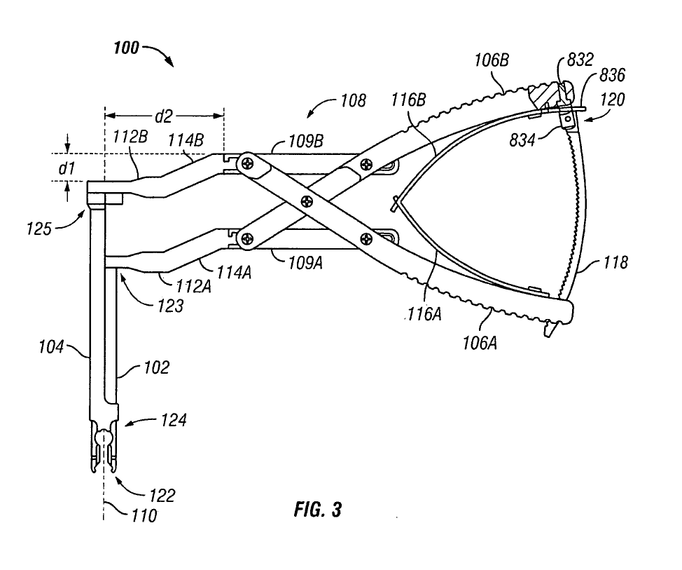

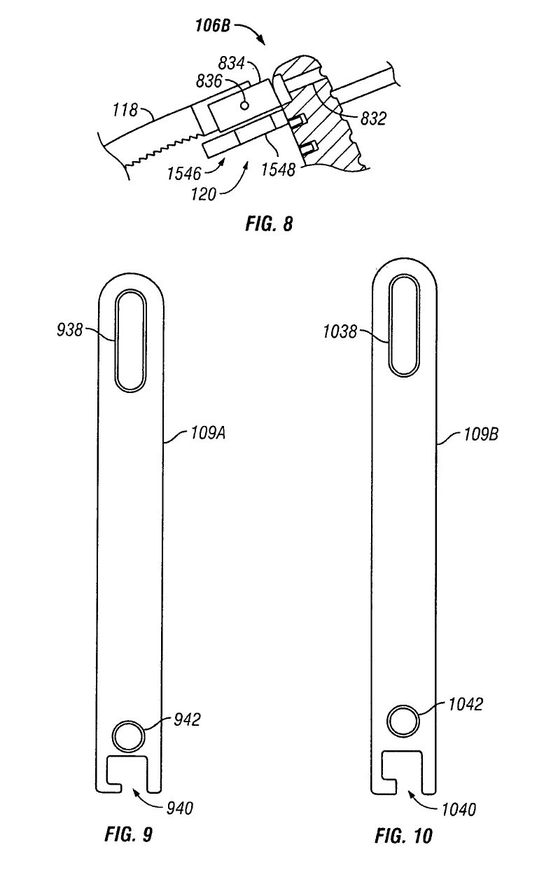

With reference now to FIG. 3, FIG. 3 illustrates a number of features of instrument 100. Among other features, FIG. 3 illustrates inner extension 102, outer extension 104, handles 106, parallel action compressor 108, female bar 109A, male bar 109B, longitudinal axis 110, offset arms 112, diagonal portions 114, biasing members 116, toothed member 118, ratchet assembly 120, distal end 122 of inner extension 102, proximal end 123 of inner extension 102, distal end 124 of outer extension 104, proximal end 125 of outer extension 104, outer ratchet holder 832, inner ratchet holder 834, and ratchet pawl 836. Ratchet components 832, 834, and 836 will be discussed further with reference to FIG. 8.

Inner extension 102 can be elongated along longitudinal axis 110 with distal end 122 being adapted to engage pedicle screw 12 (of FIG. 1) or another bone anchor system. Distal end 122 can be made of a pliant material or otherwise be configured to be pliant so that distal end 124 of outer extension 104 can radially compress distal end 122 to cause it to grasp pedicle screw 12. Distal end 122 can be adapted to receive rod 14 in a proud position relative to pedicle screw 12 via a slot or other feature. Inner extension 102 can define a cannula through which a setscrew or compression or locking member, etc. can be passed to lock rod 14 in place in pedicle screw 12. Inner extension can have a user selected length which allows surgical personnel to maneuver distal ends 122 and 124 to a surgical site where pedicle screw 12 and rod 14 may have been previously implanted.

Outer extension 104 can be elongated along longitudinal axis 110 and can define a cannula into which inner extension 102 can fit. Outer extension 104 can be coaxial with inner extension 102. The side of outer extension 104 toward parallel action compressor 108 can be open so that, when inner and outer extensions 102 and 104 translate relative to each other, offset arm 112A can move without interference from outer extension 104. Distal end 124 may also include features to compress distal end 122 of inner extension 102 to cause it to grasp pedicle screw 12 as outer extension 104 moves toward rod 14. Distal end 124 of outer extension 104 can have a groove, slot, etc. with which it can engage rod 14. In some embodiments, inner extension 102 (which can receive rod 14) can be referred to as female extension 102. Distal end 124 of outer extension 104 can be configured to urge rod 14 into position in pedicle screw 12. In some embodiments, outer extension 104 can be referred to as male extension 104. When assembled with inner extension 102, distal end 124 of outer extension 104 can be positioned generally adjacent features on inner extension 102 for receiving rod 14 as illustrated by FIG. 3. Proximal end 125 of outer extension 104 can extend beyond proximal end 123 of inner extension 102 a distance sufficient so that when surgical personnel operate instrument 100, outer extension 104 can translate relative to inner extension 102 to reduce rod 14 into position.

Proximal ends 123 and 125 of inner and outer extensions 102 and 104 can couple to offset arms 112B and 112A respectively. The coupling of proximal ends 123 and 125 to offset arms 112B and 112A can be by way of interference fits, mechanical couplings, fasteners, welds, brazes, solder, etc. Offset arms 112 can extend from inner and outer extensions 102 and 104 a distance d2 in a direction perpendicular to longitudinal axis 110. In some embodiments, offset arms 112 can include diagonal portions 114 extending in a direction which is diagonal relative to longitudinal axis 110 thereby providing an offset of distance d1 in the direction parallel to longitudinal axis 110. In some embodiments, offset distances may be defined by members having other shapes. The proximal ends of offset arms 112 can operatively couple with parallel action compressor 108 in such a manner that parallel action compressor 108 can actuate offset arms 112A and 112B. Being coupled with female (inner) extension 102, offset arm 112A can be referred to as female offset arm 112A in some embodiments. Being coupled with male (outer) extension 104, offset arm 112B can be referred to as male offset arm 112B in some embodiments. The proximal ends of offset arms 112A and 112B can couple to the distal ends of bars 109A and 109B of parallel action compressor 108.

Bars 109A and 109B can form the parallel linkages of parallel action compressor 108. Bars 109A and 109B can extend from offset arms 112A and 112B in a direction perpendicular to longitudinal axis 110 thereby further offsetting handles 106 from inner and outer extensions 102 and 104. Bar 109A can be coupled with female offset arm 112A and can be referred to as female bar 109A in some embodiments. Bar 109B can be coupled with male offset arm 112B and can be referred to as male bar 109B in some embodiments. As viewed from the side of instrument 100, offset arm 112A and bar 109A can have shapes corresponding to the shapes of offset arm 112B and bar 112B respectively and can be parallel thereto. While offset arms 112A and 112B are shown with a portion oriented diagonal to longitudinal axis 110, other configurations of offset arms 112 are envisioned. In some embodiments, offset arms 112 can be curved, be “S” shaped, have a stair-step profile when viewed from the side, etc.

Handles 106A and 106B can pivotably couple to bars 109A and 109B at the distal ends of bars 109A and 109B; slidably engage bars 109A and 109B at the proximal ends of bars 109A and 109B; and pivotably couple to each other between bars 109A and 109B (as shown in FIG. 3) to form the input linkages of parallel action compressor 108. Handles 106 can extend perpendicularly from longitudinal axis 110 a distance beyond parallel action compressor 108. Handles 106 may be curved, have knurls, and other ergonomic features to facilitate operation of instrument 100 by surgical personnel. Biasing members 116 may be coupled to handles 106 to bias handles 106 apart thereby biasing the distal end 124 of outer extension 104 away from distal end 122 of inner extension 102 via parallel action compressor 108 and offset arms 112A and 112B. Biasing members 116 can be leaf springs, coil springs, torsional springs, etc. In some embodiments, biasing members can curve an appropriate amount (or otherwise be configured) to yield a user selected biasing force over the travel span of handles 106. In some embodiments, biasing member(s) can be supplied within parallel action compressor 108 or between offset arms 112A and 112B.

Instrument 100 of FIG. 3 can also include ratchet assembly 120 including toothed member 118 and ratchet components 832, 834, and 836. Ratchet assembly can be statically coupled to one handle 106A and operatively coupled to the other handle 106B so that as surgical personnel urge handles 106A and 106B together, ratchet assembly 120 prevents handles 106 from moving apart. Instrument 100 can include features, as discussed herein, to release ratchet assembly 120 to allow handles 106 to move together.

To operate instrument 100 of FIG. 3, surgical personnel may grasp handles 106 which can be biased apart by biasing members 116. Surgical personnel may navigate distal ends 122 and 124 of inner and outer extensions 102 and 104 to a spinal stabilization system such as the one illustrated in FIG. 1. During the navigation of distal ends 122 and 124 to the surgical site, and at other times, surgical personnel can peer over, around, or beyond proximal ends 123 and 125 of inner and outer extensions 102 and 104 (due in part to offset distances d1 and d2) to view the surgical site, the spinal stabilization system, vertebrae 10, and distal ends 122 and 124 of inner and outer extensions 102 and 104. Such improved visibility provided by various embodiments can aid surgical personnel in operating more swiftly, efficiently, and accurately than here to for possible with previously available instruments.

In some scenarios, surgical personnel may have previously placed the spinal stabilization system at a surgical site on a patient's spine through a posterior incision. More particularly, surgical personnel may have implanted a number of pedicle screws 12 in vertebrae 10 and placed rods 14 in one or more pedicle screws 12 (as illustrated in FIG. 1 and FIG. 2). Surgical personnel may align distal end 122 of inner extension 102 to accept rod 14 and advance instrument 100 until rod 14 is within distal end 122 of inner extension 102. When desired, surgical personnel can peer over or around proximal ends 123 and 125 while aligning distal end 122 with rod 14. Surgical personnel may advance distal end 122 to pedicle screw 12, place distal end 122 thereon, and begin pressing handles 106 together. Again, surgical personnel can view the surgical site all the while if desired.

Distal end 124 of outer extension 104 may advance toward distal end 122 of inner extension 102 via the action of parallel action compressor 108 as transmitted to inner and outer extensions 102 and 104 by offset arms 112. As distal end 124 of outer extension 104 advances toward and over distal end 122 of inner extension 102, features on distal end 124 can radially compress distal end 122 causing it to grip pedicle screw 12. Features of distal end 124 can engage rod 14 pushing it toward and into pedicle screw 12. Ratchet pawl 836 of ratchet assembly 120 can slidably engage teeth of toothed member 118 as handles 106 move toward each other. If surgical personnel release pressure on handles 106, biasing members 116 can urge handles 106 apart until a tooth on toothed member 118 engages ratchet pawl 836 thereby maintaining handles 106 and distal ends 122 and 124 of inner and outer extensions 102 and 104 in substantially the same position they were in when the pressure on handles 106 was released.

Surgical personnel can resume (or continue, as the case may be) pressing handles 106 together, advancing distal end 124 over distal end 122, and seating rod 14 within pedicle screw 12. As stated previously, surgical personnel can view the surgical site and visually confirm that rod 14 is seated in pedicle screw 12. If for some reason, rod 14 has not seated properly in pedicle screw 12, surgical personnel can release ratchet assembly 120 by pressing on biasing member extension 1546 (discussed with reference to FIG. 15), translate distal end 124 of outer extension 104 away from pedicle screw 12 and again attempt to seat rod 14 in pedicel screw 12.

With previously available instruments engaged with pedicle screw 12 and rod 14, it could happen that surgical personnel might not have been able to verify proper seating of rod 14 in pedicle screw 12 because handles, extensions, or other portions of these instruments obstructed the surgical personnel's view of the surgical site. In some scenarios, surgical personnel could only confirm the seating of rod 14 in pedicle screw 12 indirectly such as be tactile sensations transmitted from pedicle screw 12 and rod 14 through the instrument. As a result, in some scenarios, rod 14 might have been left in an improper position such as being proud of its intended position in pedicle screw 12 by some distance. In some scenarios, surgical personnel had to disengage previously available instruments from pedicle screw 12 and rod 14 to verify proper alignment therebetween.

If surgical personnel determined that pedicle screw 12 and rod 14 were misaligned, little could be done to correct the situation other than manually disengaging rod 14 from pedicle screw 12. Such manual intervention sometimes necessitated enlarging the incision to gain greater access to the surgical site and, more particularly, pedicle screw 12 and rod 14. Enlarging the incision and prying rod 14 from pedicle screw 14, in some scenarios, could damage pedicle screw 12 and/or rod 14, increase trauma to the surgical site, prolong surgery (and the need for local and/or general anesthesia, cause greater patient discomfort, and prolong recovery time. In some scenarios, surgical personnel might desire instead to leave pedicle screw 12 and rod 14 misaligned resulting in less than optimal performance of the spinal stabilization system and a diminishment of the relief from the condition being treated which had been sought by the patient. These results can be avoided if surgical personnel use rod reduction instruments 100 constructed in accordance with various embodiments.

With rod 14 confirmed as being seated in pedicle screw 12, surgical personnel can release pressure on handles 106A and 106B of instrument 100 thereby allowing ratchet assembly 120 to maintain distal ends 122 and 124 of inner and outer extensions 102 and 104, and therefore pedicle screw 12 and rod 14, in position relative to one another for as long as it takes for surgical personnel to confirm proper alignment of pedicle screw 12 and rod 14 or as long as desired. When desired, surgical personnel can then advance a set screw or compression or locking member through the cannula of inner extension 102. Surgical personnel can use the compression member to lock rod 14 in its desired and perhaps confirmed position in pedicel screw 12. Surgical personnel can then remove any instrument or locking driver (with which they locked the compression member in place) from the cannula of inner extension 102. If desired, surgical personnel can release ratchet assembly 120 and draw handles 106 apart, thereby withdrawing distal end 124 of outer extension 104 from distal end 122 of inner extension 102. As distal end 124 withdraws, it allows distal end 122 to expand radially, thereby releasing the grasp of distal end 122 from pedicle screw 12. Surgical personnel may then withdraw instrument 100 from the surgical site and close the same.

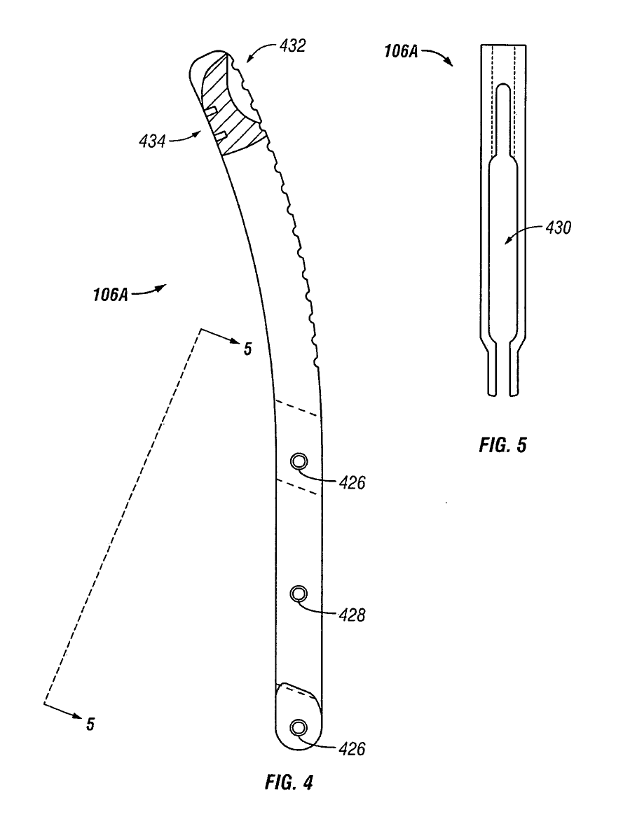

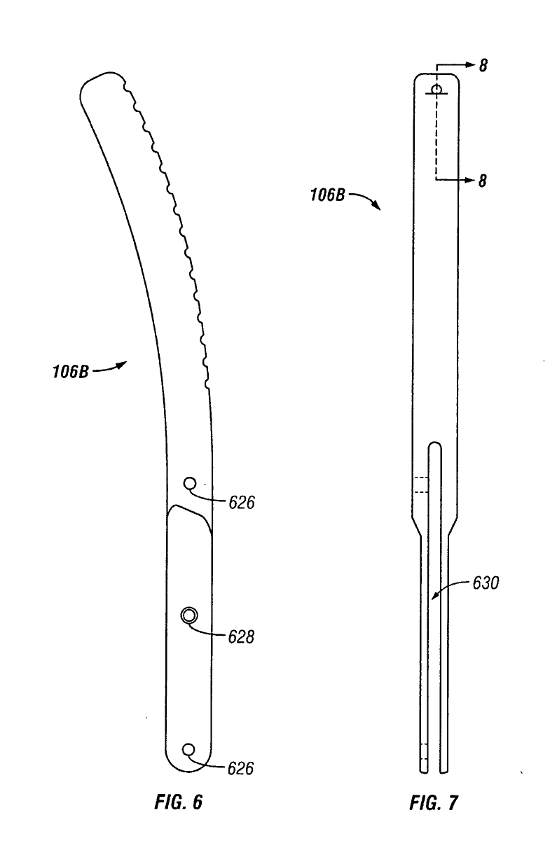

With reference now to FIGS. 4-7, handles 106A and 106B can include pairs of coupling points 426 and 626 on the portions of handles 106A and 106B, respectively, which can pivotably couple with bars 109A and 109B or slidably engage bars 109A and 109B as the case may be (handles 106A and 106B are illustrated pivotably coupled to bars 109A and 109D in FIG. 3). Handles 106A and 106B can also include coupling points 428 and 628 in the portions of handles 106A and 106B where handles 106A and 106B pivotably couple to each other. Coupling points 426, 428, 626, and 628 can be adapted to receive pins, bolts, tabs, projections, etc. as may be appropriate for the type of engagement (pivotable coupling, sliding engagement, etc.) which coupling points 426, 428, 626, and 628 facilitate between handles 106A and 106B and bars 109A and 109B and between handles 106A and 106B themselves. With particular reference to FIG. 4, handle 106A can have a thumb grip 432 or other ergonomic features to facilitate surgical personnel grasping and actuating instrument 100. Handle 106A can also include features 434 to fixedly attached toothed member 118 (see FIG. 3) to handle 106A. Features 434 can be fasteners, detents, etc.

Handles 106A and 106B can also include slots 430 and 630 as shown in FIGS. 5 and 7. Slots 430 and 630 can be shaped to accommodate the movements of handles 106A and 106B and bars 109A and 109B as surgical personnel move handles 106A and 106B to operate instrument 100. Slot 630 of handle 106A can have a generally uniform cross section (as seen from above in FIGS. 1-3) to allow one a portion of handle 106A to pass partially through handle 106B as handles 106A and 106B are actuated. Slot 430 of handle 106A can allow a portion of handle 106B to pass partially through handle 106A as handles 106A and 106B are actuated. Slot 430 can have an enlarged portion through which a portion of bars 109A and 109B can pass as handles 106A and 106B are actuated.

Ratchet assembly 120 is further illustrated in FIG. 8. FIG. 8 shows the proximal end of handle 106B to which ratchet assembly 120 can be operatively coupled in some embodiments. Ratchet assembly 120 can include outer ratchet holder 832, inner ratchet holder 834, and ratchet pawl 836. Ratchet pawl 836 can be a pin, small rod, tab, etc. As will be discussed herein, ratchet assembly 120 can cooperate with toothed member 118 and extension 1546 (see FIG. 15) of biasing member 116B to allow handles 106 to ratchet apart from each other, Biasing member extension 1546 can extend beyond the proximal end of handle 106B and engage ratchet pawl 836. Biasing member 116B can include pawl release feature 1548 on extension 1546 (see FIG. 15). Toothed member 118 can extend to, or beyond, handle 106B and ratchet assembly 120. Toothed member 118 may have a radius of curvature corresponding to the location of ratchet pawl 836 relative to coupling points 428 and 628 and on handle 106B (see FIG. 3) so that when handles 106 pivot relative to each other, toothed member 118 remains engaged with ratchet pawl 836. Biasing members 116, by biasing handles 106A and 106B apart in some embodiments, can assist with keeping ratchet pawl 836 engaged with various teeth of toothed member 118.

Outer ratchet holder 832 can couple ratchet assembly 120 to handle 106B. Inner ratchet assembly 834 can couple to outer ratchet holder 832 and provide guides for toothed member 118 and extension 1346. In operation, as surgical personnel urge handles 106A and 106B together, ratchet pawl 836 slidably engages various teeth of toothed member 118. When handles 106A and 106B begin to move apart, ratchet pawl 836 engages the tooth upon which it rests in such a manner that ratchet pawl 836 prevents movement of toothed member 118 and, hence, handles 106A and 106B. When handles 106A and 106B again move toward each other, ratchet pawl 836 can again slidably engage various teeth of toothed member 118. When desired, surgical personnel may urge pawl release feature 1548 into contact with the portion of toothed member currently beside ratchet assembly 120. Doing so can cause pawl release feature 1548 to urge toothed member 118 away from ratchet assembly 120, thereby releasing ratchet pawl 836 from engagement with toothed member 118.

FIGS. 9 and 10 illustrate bars 109A and 109B which can form parallel linkages of parallel action compressor 108. Bars 109A and 109B can include slots 938 and 1048 for slidably engaging handles 106A and 106B with any of various pins, bolts, tabs, projections, etc. In some embodiments, bars 109A and 10B include attachments points 940 and 1040 for coupling handles 106A and 106B to bars 109A and 109B. Attachment points 942 and 1042 of FIGS. 9 and 10 can be adapted to receive pins, bolts, tabs, projections, etc. to pivotably couple handles 106A and 106B and bars 109A and 109B. Since bars 109A and 109B couple indirectly with female (inner) extension 102 and male (outer) extension 104 through offset arms 112A and 112B, bars 109A and 109B can be referred to as female bar 109A and male bar 109B. Bars 109A and 109B can be straight with generally rectangular cross sections generally free of features which might interfere with handles 106A and 106B as handles 106A and 106B are actuated.

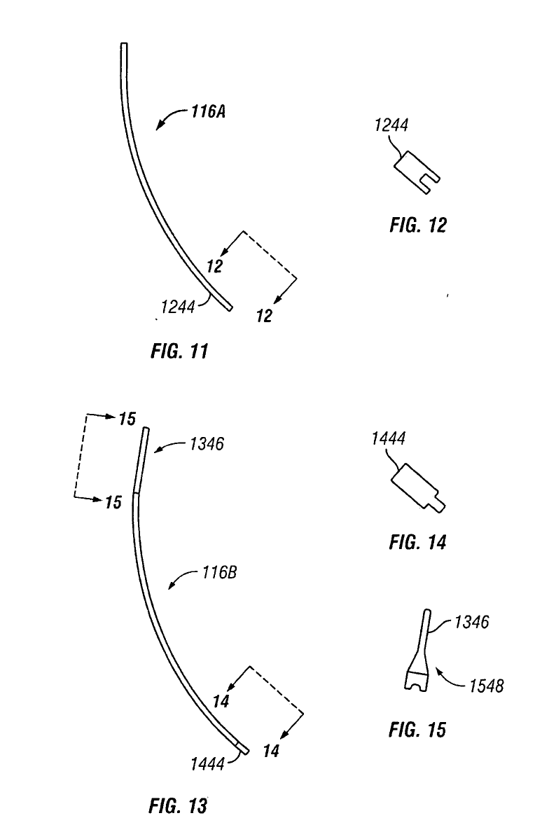

FIGS. 11-15 illustrate biasing members 116. Biasing member 116A can couple to handle 106A at one end and to biasing member 116B at the other end. Biasing member can couple to handle 106B at one end and to biasing member 116A at the other end. The coupling of biasing members 116 to handles 106A and 106B can be by any number of coupling techniques including, but not limited to, screws, bolts, pop rivets, brazes, welds, clamps, etc. In some embodiments, biasing members 116A and 116B can include couplings 1244 and 1444 (of FIGS. 12 and 14) at their ends opposite the coupling to handles 106A and 106B. Couplings 1244 and 1444 can be used to couple biasing members 116A and 116B to each other. In some embodiments, couplings 1244 and 1444 can disengage from each other to release the biasing force on handles 106A and 106B caused by biasing members 116.

With more particular reference to FIGS. 13 and 15, biasing member 116B can include extension 1546. Extension 1546 can be a straight portion of biasing member 116B which, when assembled into instrument 100, can extend beyond ratchet assembly 120 (see FIG. 8) and beyond the proximal end of handle 106B. Extension 1546 can include pawl release feature 1548 (see FIG. 15) so that by pressing on extension 1546, surgical personnel can urge biasing member 116 away from the proximal end of handle 106B and ratchet pawl 836 thereby releasing ratchet assembly 120 (see FIG. 8) and allowing handles 106A and 106B to be drawn apart if desired. Surgical personnel can remove pressure from ratchet release feature 1548 to allow toothed member 118 to re-engage ratchet assembly 120 and ratchet pawl 836. With toothed member 118 re-engaged with ratchet pawl 836, surgical personnel can compress handles 106A and 106B toward each other to actuate inner and outer extensions 102 and 104 but cannot draw handles 106A and 106B apart to withdraw outer extension 104 from inner extension 102.

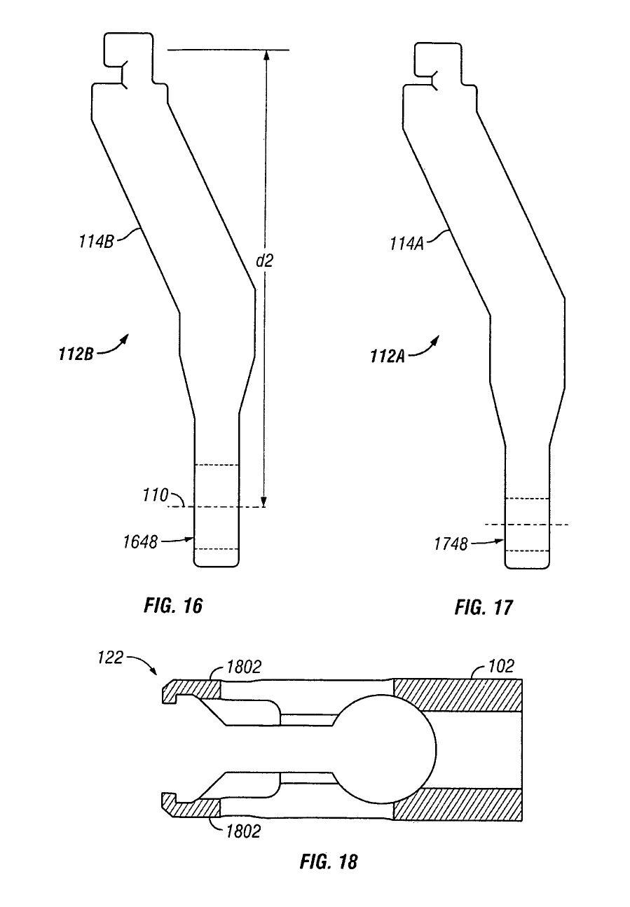

With reference now to FIGS. 16 and 17, offset arms 112A and 112B are further illustrated by FIGS. 16 and 17. Offset arms 112A and 112B can be similar to each other except for features to accommodate the differences between inner and outer extensions 102 and 104. Offset arms 112A and 112B can include couplings 1650 and 1750 for coupling with corresponding features on bars 109A and 109B. Offset arms 112A and 112B can define apertures 1648 and 1748. Apertures 1648 and 1748 can be shaped to cause an interference fit with inner and outer extensions 102 and 104 thereby coupling inner and outer extensions 104 and 102 to offset arms 112B and 112A respectively. Offset arms 112A and 112B can, in some embodiments, define gussets, ridges flanges, etc. adjacent to, or within, apertures 1648 and 1748 for retaining inner and outer extensions 102 and 104 particularly when surgical personnel exert force on handles 106 to urge rod 14 into pedicle screw 12 (see FIG. 2). Aperture 1648 can, in conjunction with the cannula of inner extension 102, allow surgical personnel to place a set screw or locking or compression member on pedicle screw 12 and rod 14 (see FIG. 2). Surgical personnel can translate a locking driver through aperture 1648 and inner extension 102 cannula to lock rod 14 in its desired position in pedicle screw 12.

With reference now to FIG. 18, FIG. 18 illustrates one embodiment of distal end 122 of inner extension 102. Distal end 122 can include resilient fingers 1802 which can be adapted to receive rod 14 (of FIG. 2). Resilient fingers 1802 can be sized and configured such that, as outer extension 104 translates toward and over distal end 122, outer extension 104 can compress resilient fingers 1802 in toward pedicle screw 12 (see FIG. 2). Resilient fingers 1802 can be biased to expand back to the position shown in FIG. 18 as outer extension withdraws from distal end 122. Resilient fingers 1802 can therefore grasp pedicle screw 12 and release it depending on the position of outer extension 104. While FIG. 18 shows resilient fingers 1802, some embodiments include other features for holding pedicle screw 12 in position relative to distal end 122. Such features can include threads, detents, bayonet type fittings, etc. without departing from the scope of the disclosure.

Although embodiments have been described in detail herein, it should be understood that the description is by way of example only and is not to be construed in a limiting sense. It is to be further understood, therefore, that numerous changes in the details of the embodiments and additional embodiments will be apparent, and may be made by, persons of ordinary skill in the art having reference to this description. It is contemplated that all such changes and additional embodiments are within scope of the claims below and their legal equivalents.

1. An instrument for the reduction of a rod into position in a pedicle screw, the instrument comprising:
an inner extension having proximal and distal ends, the distal end of the inner extension adapted to engage the pedicle screw;
an outer extension positioned about the inner extension and having proximal and distal ends and a longitudinal axis, the distal end of the outer extension being adapted to engage the rod;
a first handle having proximal and distal ends, the distal end of the first handle being coupled with the proximal end of the inner extension; and
a second handle having proximal and distal ends, the distal end of the second handle being coupled with the proximal end of the outer extension, the handles extending generally perpendicular from the respective extensions and being operatively coupled to form an actuator for urging the extensions in a direction relative to each other along the longitudinal axis, the handles being offset from the proximal end of at least one of the extensions.

2. The instrument of claim 1 wherein the offset is in a direction parallel to the longitudinal axis.

3. The instrument of claim 2 wherein the offset is greater than about half inch.

4. The instrument of claim 1 wherein the offset is in a direction perpendicular to the longitudinal axis.

5. The instrument of claim 4 wherein the offset is greater than about two and one half inches.

6. The instrument of claim 1 further comprising a locking mechanism extending between the first and second handles.

7. The instrument of claim 6 wherein the locking mechanism includes a ratchet with a toothed member extending between the first and second handles, and a pawl operatively coupled to one of the handles for engaging teeth of the toothed member to prevent the handles from moving apart from one another.

8. The instrument of claim 7 further comprising a biasing member coupled to the first and the second handles, the biasing member positioned to urge the first and second handles apart.

9. The instrument of claim 8 wherein the biasing member is a leaf spring.

10. The instrument of claim 8 wherein the biasing member is positioned to urge the teeth and pawl to engage.

11. The instrument of claim 9 further comprising an extension of the leaf spring beyond the proximal end of one of the handles, the extension being adapted to disengage the teeth and the pawl.

12. The instrument of claim 1 wherein the inner extension defines a passage for allowing a compression member to be inserted through the passage.

13. The instrument of claim 1 wherein the actuator further comprises a parallel action compressor.

14. A kit for stabilizing a portion of a spine comprising:
a set of pedicle screws of various configurations;
a set of rods of various configurations; and
an instrument for the reduction of at least one of the rods into position in at least one of the pedicle screws, the instrument further comprising:
an inner extension having proximal and distal ends, the distal end of the inner extension adapted to engage the pedicle screw;
an outer extension positioned about the inner extension and having proximal and distal ends and a longitudinal axis, the distal end of the outer extension being adapted to engage the rod;
a first handle having proximal and distal ends, the distal end of the first handle being coupled with the proximal end of the inner extension; and
a second handle having proximal and distal ends, the distal end of the second handle being coupled with the proximal end of the outer extension, the handles extending generally perpendicular from the respective extensions and being operatively coupled to form an actuator for urging the extensions in a direction relative to each other along the longitudinal axis, the handles being offset from the proximal end of at least one of the extensions.

15. The kit of claim 14 further comprising a set of set of setscrews of various configuration, the setscrews for locking the rod in position in the pedicle screw wherein the inner extension has a cannulation for accepting the setscrew.

16. The kit of claim 14 further comprising a locking driver for locking the rod in position in the pedicle screw using a setscrew wherein the inner extension has a cannulation for accepting the locking driver.

17. The kit of claim 14 wherein the offset is in a direction selected from the group consisting of a direction parallel to the longitudinal axis and a direction perpendicular to the longitudinal axis.

18. The kit of claim 14 wherein the instrument includes a ratchet with a toothed member extending between the first and second handles and a pawl operatively coupled to one of the handles for engaging teeth of the toothed member to prevent the handles from moving apart from one another.

19. The kit of claim 18 wherein the instrument further includes a biasing member coupled to the first and the second handles, the biasing member positioned to urge the first and second handles apart.

20. The kit of claim 18 wherein the biasing member is a leaf spring and is further positioned to urge the teeth and pawl to engage, the instrument further comprising an extension of the leaf spring beyond the proximal end of one of the handles, the extension being adapted to disengage the teeth and the pawl.

21. An instrument for the reduction of a rod into position in a pedicle screw, the instrument comprising:
a female extension having proximal and distal ends and defining a passage for allowing a compression member to be inserted through the passage, the distal end of the female extension adapted to engage the pedicle screw;
a male extension positioned about the female extension and having proximal and distal ends and a longitudinal axis, the distal end of the male extension being adapted to engage the rod;
a first handle having proximal and distal ends, the distal end of the first handle being coupled with the proximal end of the female extension;
a second handle having proximal and distal ends, the distal end of the second handle being coupled with the proximal end of the male extension, the handles extending generally perpendicular from the respective extensions and being operatively coupled to form a parallel action compressor for urging the extensions in a direction relative to each other along the longitudinal axis, the handles being offset from the proximal end of at least one of the extensions;
a ratchet mechanism with a toothed member extending between the first and second handles and a pawl operatively coupled to one of the handles for engaging teeth of the toothed member to prevent the handles from moving apart from one another;
at least one leaf spring coupled to the first and the second handles, the leaf spring positioned to urge the first and second handles apart and to urge the teeth and pawl to engage; and
an extension of the leaf spring beyond the proximal end of one of the handles, the extension being adapted to disengage the teeth and the pawl.
